(19) United States
(12) Patent Application Publication
(10) Pub. No.: US 2023/0000832 A1
(43) Pub. Date: Jan. 5, 2023

(54) USE OF NEUROPILIN ANTAGONISTS FOR THE TREATMENT OF ENDOMETRIOSIS

(71) Applicants: INSERM (Institut National de la Santé et de la Recherche Médicale), Paris (FR); Fondation Imagine, Paris (FR); Université Paris Cité, Paris (FR); Assistance Publique-Hôpitaux de Paris (APHP), Paris (FR)

(72) Inventors: Yves LEPELLETIER, Paris (FR); Jean GOGUSEV, Paris (FR); Rachel RIGNAULT-BRICARD, Paris (FR); Nicolas LOPEZ, Paris (FR)

(21) Appl. No.: 17/779,833

(22) PCT Filed: Nov. 27, 2020
(86) PCT No.: PCT/EP2020/083638

§ 371 Date: May 25, 2022

(30) Foreign Application Priority Data
Nov. 27, 2019 (EP) 19306530.7

(51) Int. Cl. A61K 31/4184; A61K 31/56; A61P 15/00

(52) U.S. Cl. CPC A61K 31/4184; A61K 31/56; A61P 15/00

(57) ABSTRACT

Endometriosis is a chronic inflammatory systemic sex hormone-dependent gynecological disease, characterized by the presence and growth of endometrial tissue (glands and stroma) outside the uterine cavity, predominantly, but not exclusively, in the pelvic compartment. Here, the inventors show that the use of Neuropilin/VEGF binding inhibitors, so called, Neuropilin antagonist (NRPa), bring new perspective to treat and cure endometriosis in women suffering thereof. NRPa alone is efficient to inhibit primary endometrial cell proliferation and apoptosis/necrosis program cell death of targeted cells. The effective NRPa concentration needed is very low (NRPa-48 IC₅₀=10⁻⁷M) and is dependent of the NRPa structure. Moreover, the association of NRPa with progestogen drug increases the anti-proliferative effect. Therefore, the present invention relates to the use of neuropilin antagonists for the treatment of endometriosis.

### FIELD OF THE INVENTION

The present invention is in the field of medicine, in particular gynaecology.

### BACKGROUND OF THE INVENTION

Endometriosis is a chronic inflammatory systemic sex hormone-dependent gynecological disease, characterized by the presence and growth of endometrial tissue (glands and stroma) outside the uterine cavity, predominantly, but not exclusively, in the pelvic compartment. This growth, so-called a lesion, can occur on reproductive organs but rare endometriosis form (20% of cases) exist. This rare form, so-called extrapelvic form, is characterized by the presence of functional endometrial glands and stroma in several locations, such as lungs, pleura, kidneys, bladder, abdominal wall, umbilicus, and cesarean section scar (cutaneous endometriosis form) among others as classified by the Orphanet rare disease classification (ORPHA:137820).

This disease affects at least 10% of women of reproductive age (approximatively 300 millions of women around the world) with a 0.1% annual incidence of endometriosis among women aged 15-49 years and with a peak between 25 years and 35 years of age. [Parazzini, F., Vercellini, P. & Pelucchi, C. in Endometriosis science and practice (eds Giudice, L. C., Johannes, L. H. & Healy, D. L.) 19-26 (Wiley-Blackwell Publishing, 2012)] [Viganò, P., Parazzini, F., Somigliana, E. & Vercellini, P. Endometriosis: epidemiology and aetiological factors. Best Pract. Res. Clin. Obstet. Gynaecol. 18, 177-200 (2004)]. This disease is associated to pelvic pain and infertility [L. C. Giudice, “Clinical practice. Endometriosis,” New England Journal of Medicine, vol. 362, no. 25, pp. 2389-2398, 2010] [S. E. Bulun, “Endometriosis,” New England Journal of Medicine, vol. 360, no. 3, pp. 268-279, 2009].

Clinical manifestations are menstrually-related and depend on the location of the ectopic tissue, but in general include pain, mass/nodule, swelling and/or bleeding in the involved area.

Three different phenotypes are recognized including superficial, ovarian endometrioma and deep infiltrating endometriosis. The disease is associated with adenomyosis in approximately 30% of the patients. This disease should be considered as an important public health problem having a major effect on the quality of women's life as being long time a substantial economic burden. Medical treatment is the first line therapeutic option for patients with pelvic pain and no desire for immediate pregnancy. In patients with infertility, careful consideration should be made regarding whether to provide assisted reproductive technologies prior to performing endometriosis surgery. Hormonal medication that suppresses the natural cycle, when combined with pain medication, is the typical treatment. These previous hormonal treatments of endometriosis have focused on shutting down estradiol production from the ovary. The failure of this direction of therapy is that it does not suppress estrone from the adrenal glands or environmental toxins (xeno-estrogens) leaking out of the adipose tissue.

Among the hormones which have been used to treat endometriosis are progestogens oral contraceptives and the like modern endometriosis management should be individualized with a patient centered multi modal and interdisciplinary integrated approach.

Therapeutically, there main options exist for endometriosis treatment, including medical treatment, surgery and assisted reproductive technology (ART).

Hormonal treatment widely used are effective for the treatment of the symptoms but are not curative, that is, they relieve pain without eliminating the endometriotic lesions. For patients who do not respond to hormonal therapy, new drugs are urgently needed. Novel emerging drugs including GnRH antagonists, selective oestrogen or progesterone receptor modulators, anti-angiogenic drugs, anti-oxydants, immunomodulators as well as certain epigenetic agents represent a new promising treatment, though all require more thorough experimental and clinical evaluation.

### SUMMARY OF THE INVENTION

As defined by the claims, the present invention relates to the use of neuropilin antagonists for the treatment of endometriosis.

### DETAILED DESCRIPTION OF THE INVENTION

Here, the inventors show that the use of Neuropilin/VEGF binding inhibitors, so called, Neuropilin antagonist (NRPa), bring new perspective to treat and cure endometriosis in women suffering thereof. NRPa alone is efficient to inhibit primary endometrial cell proliferation and apoptosis/necrosis program cell death of targeted cells. The effective NRPa concentration needed is very low (NRPa-48 IC₅₀=10⁻⁷M) and is dependent of the NRPa structure. Moreover, the association of NRPa with progestatif drug increase the anti-proliferatif effect.

Thus, the first object of the present invention relates to a method of treating endometriosis in a patient in need thereof comprising administering to the patient a therapeutically effective amount of a neuropilin antagonist (NRPa).

As used herein, the term “endometriosis” has its general meaning in the art (see Background section”. In particular, the term includes peritoneal endometriosis, ovarian endometriosis, deep endometriosis and extrapelvic endometriosis.

As used herein the term “patient” refers to a mammalian female to which the present invention may be applied. Typically said mammal is a human (i.e. a woman), but may concern other mammals such as primates, dogs, cats, pigs, sheep, cows.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]). In particular, in the case of AMLs, maintenance therapy may eradicate clinically invisible minimal residual disease.

More particularly, the term “treatment of endometriosis” includes treatment to reduce (or remove) the amount of endometrial tissue which is present inside and/or outside the uterine cavity (e.g. reduction or removal of endometriotic lesions); and/or treatment to reduce and/or ameliorate one or more symptoms associated with endometriosis (e.g. treatment to ameliorate and/or reduce the symptoms of dysmenorrhoea; treatment to ameliorate and/or reduce the symptoms of dyspareunia, and/or treatment to ameliorate and/or reduce pelvic pain). The American Society for Reproductive Medicine (ASRM) defines a classification system for the various stages of endometriosis, dividing this into four stages (stage IV most severe; stage I least severe) [American Society for Reproductive Medicine. Revised American Society for Reproductive Medicine classification of endometriosis: 1996. Fertil Steril 1997; 67,817 821.]. Thus, the term “treatment of endometriosis” includes treatment to reduce the severity of the condition as measured by ASRM classification, e.g. treatment to reduce the severity of the endometriosis from Stage IV, III, II, or I to a lower stage, or until the symptoms are completely alleviated.

As used herein, the term “neuropilin” or “NRP” has its general meaning in the art and refers to a transmembrane glycoprotein that typically consists of five domains: three extracellular domains (a1 a2, b1, b2 and c), a transmembrane domain and a cytoplasmic domain. There are two neuropilin members: neuropilin-1 (NRP-1) and neuropilin-2 (NRP-2) that are share 44% sequence homology. Neuropilins are multifunctional non-tyrosine kinase receptors for some members of VEGF (Vascular Endothelial Growth Factor) family members, including VEGF-A₁₆₅ and for others ligands such as, but not limited to class 3 semaphorin, TGF-β (transforming growth factor beta-1), HGF (hepatocyte growth factor), FGF (fibroblast growth factor), and PDGF (platelet-derived growth factor) [Neuropilin-1 as therapeutic target for malignant melanoma. Front. Oncol., 3 Jun. 2015 | https://doi.org/10.3389/fonc.2015.00125].

As used herein, the term “neuropilin antagonist” refers to a molecule that partially or fully blocks, inhibits, or neutralizes a biological activity or expression of a neuropilin protein. A neuropilin antagonist can be a molecule of any type that interferes with the signalling associated with at least one or more neuropilin family members (e.g. NRP-1 or NRP-2) in a cell, for example, either by decreasing transcription or translation of neuropilin-encoding nucleic acid, or by inhibiting or blocking neuropilin polypeptide activity, or both. Examples of neuropilin antagonists include, but are not limited to, antisense polynucleotides, interfering RNAs, catalytic RNAs, RNA-DNA chimeras, neuropilin-specific aptamers, anti-neuropilin antibodies, neuropilin-binding fragments of anti-neuropilin antibodies, neuropilin-binding small molecules, neuropilin-binding peptides, and other polypeptides that specifically bind neuropilin (including, but not limited to, neuropilin-binding fragments of one or more neuropilin ligands, optionally fused to one or more additional domains), such that the interaction between the neuropilin antagonist and neuropilin results in a reduction or cessation of neuropilin activity or expression. In particular, neuropilin antagonist inhibits the interaction between a neuropilin protein (e.g. NRP-1) and one of its partners, in particular VEGF-A₁₆₅.

Neuropilin antagonists are well known in the art and typically include those describe in:
Liu W Q, Megale V, Borriello L, Leforban B, Montes M, Goldwaser E, Gresh N, Piquemal J P, Hadj-Slimane R, Hermine O, Garbay C, Raynaud F, Lepelletier Y, Demange L. Synthesis and structure-activity relationship of non-peptidic antagonists of neuropilin-1 receptor. Bioorg Med Chem Lett. 2014 Sep. 1; 24(17):4254-9.
Tymecka D, Puszko A K, Lipinski P F J, Fedorczyk B, Wilenska B, Sura K, Perret G Y, Misicka A. Branched pentapeptides as potent inhibitors of the vascular endothelial growth factor 165 binding to Neuropilin-1: Design, synthesis and biological activity. Eur J Med Chem. 2018 Oct. 5; 158:453-462.
Liu W Q, Lepelletier Y, Montes M, Borriello L, Jarray R, Grépin R, Leforban B, Loukaci A, Benhida R, Hermine O, Dufour S, Pagès G, Garbay C, Raynaud F, Hadj-Slimane R, Demange L. NRPa-308, a new neuropilin-1 antagonist, exerts in vitro anti-angiogenic and anti-proliferative effects and in vivo anti-cancer effects in a mouse xenograft model. Cancer Lett. 2018 Feb. 1; 414:88-98.
Borriello L, Montes M, Lepelletier Y, Leforban B, Liu W Q, Demange L, Delhomme B, Pavoni S, Jarray R, Boucher J L, Dufour S, Hermine O, Garbay C, Hadj-Slimane R, Raynaud F. Structure-based discovery of a small non-peptidic Neuropilins antagonist exerting in vitro and in vivo anti-tumor activity on breast cancer model. Cancer Lett. 2014 Jul. 28; 349(2):120-7.
Getz J A, Cheneval O, Craik D J, Daugherty P S. Design of a cyclotide antagonist of neuropilin-1 and -2 that potently inhibits endothelial cell migration. ACS Chem Biol. 2013; 8(6):1147-54.
Jia H, Bagherzadeh A, Hartzoulakis B, Jarvis A, Lohr M, Shaikh S, Aqil R, Cheng L, Tickner M, Esposito D, Harris R, Driscoll P C, Selwood D L, Zachary I C. Characterization of a bicyclic peptide neuropilin-1 (NP-1) antagonist (EG3287) reveals importance of vascular endothelial growth factor exon 8 for NP-1 binding and role of NP-1 in KDR signaling. J Biol Chem. 2006 May 12; 281(19):13493-502.
A. Starzec, P. Ladam, R. Vassy, S. Badache, N. Bouchemal, A. Navaza, C. H. du Penhoat and G. Y. Perret, Structure-function analysis of the antiangiogenic ATWLPPR peptide inhibiting VEGF(165) binding to neuropilin-1 and molecular dynamics simulations of the ATWLPPR/neuropilin-1 complex, Peptides 28 (2007) 2397-402.
A. Novoa, N. Pellegrini-Moise, D. Bechet, M. Barberi-Heyob and Y. Chapleur, Sugar-based peptidomimetics as potential inhibitors of the vascular endothelium growth factor binding to neuropilin-1, Bioorg. Med. Chem 18. (2010) 3285-98.
C. Nasarre, M. Roth, L. Jacob, L. Roth, E. Koncina, A. Thien, G. Labourdette, P. Poulet, P. Hubert, G. Crémel, G. Roussel, D. Aunis, D. Bagnard, Peptide-based interference of the transmembrane domain of neuropilin-1 inhibits glioma growth in vivo, Oncogene 29 (2010) 2381-92.

In some embodiments, the neuropilin antagonist is an antibody that specifically binds to a neuropilin (e.g. NRP-1 or NRP-2) and neutralizes its activity to activate neuropilin signalling pathway, and in particular inhibits the binding neuropilin and VEGF-A₁₆₅. In some embodiments, the antibody binds to an extracellular domain of neuropilin. In some embodiments, the antibody binds to the domain c of NRP-1. Examples of antibodies that are neuropilin antagonists include those described in WO2011/143408 that described in particular the anti-NRP-1 antibody MNRP1685A.

As used herein, the term “antibody” as includes but is not limited to polyclonal, monoclonal, humanized, chimeric, Fab fragments, Fv fragments, F(ab′) fragments and F(ab′)2 fragments, as well as single chain antibodies (scFv), fusion proteins and other synthetic proteins which comprise the antigen-binding site of the antibody. Antibodies can be made by the skilled person using methods and commercially available services and kits known in the art. Methods of preparation of monoclonal antibodies are well known in the art and include hybridoma technology and phage display technology. Further antibodies suitable for use in the present disclosure are described, for example, in the following publications: Antibodies A Laboratory Manual, Second edition. Edward A. Greenfield. Cold Spring Harbor Laboratory Press (Sep. 30, 2013); Making and Using Antibodies: A Practical Handbook, Second Edition. Eds. Gary C. Howard and Matthew R. Kaser. CRC Press (Jul. 29, 2013); Antibody Engineering: Methods and Protocols, Second Edition (Methods in Molecular Biology). Patrick Chames. Humana Press (Aug. 21, 2012); Monoclonal Antibodies: Methods and Protocols (Methods in Molecular Biology). Eds. Vincent Ossipow and Nicolas Fischer. Humana Press (Feb. 12, 2014); and Human Monoclonal Antibodies: Methods and Protocols (Methods in Molecular Biology). Michael Steinitz. Humana Press (Sep. 30, 2013)).

In some embodiments, the neuropilin antagonist is a small molecule, such as a small organic molecule, which typically has a molecular weight less than 5,000 kDa.

Examples of small molecules that are neuropilin antagonists include those described in WO2012156289 that are:
N-[5-(1H-benzimidazol-2-yl)-2-methylphenyl]-N′-(2,3-dihydro-1,4-benzodioxin-6- ylcarbonyl)thiourea (also named NRPa-47):

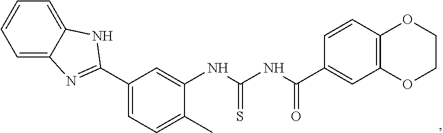 [SMILES: Cc1ccc(-c2nc3ccccc3[nH]2)cc1NC(=S)NC(=O)c1ccc2c(c1)OCCO2]

N-[3-(1H-benzimidazol-2-yl)phenyl]-N′-(2,3-dihydro-1,4-benzodioxin-6-ylcarbonyl)thiourea (also named NRPa-48):

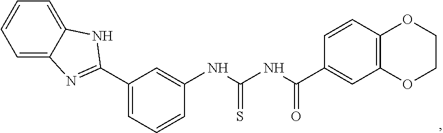 [SMILES: O=C(NC(=S)Nc1cccc(-c2nc3ccccc3[nH]2)c1)c1ccc2c(c1)OCCO2]

and/or
N-[3-(1H-benzimidazol-2-yl)phenyl]-N′-(1,3-benzodioxol-5-ylcarbonyl)thiourea

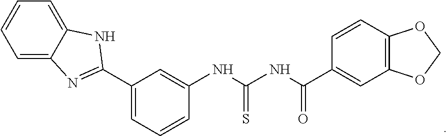 [SMILES: O=C(NC(=S)Nc1cccc(-c2nc3ccccc3[nH]2)c1)c1ccc2c(c1)OCO2]

or their salts and esters, and mixtures thereof.

Another example includes N-(2-ethoxyphenyl)-4-methyl-3-(N-(p-tolyl)sulfamoyl)benzamide that has been described in WO2015004212 and having the formula of

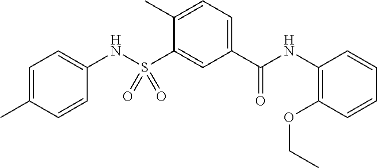 [SMILES: CCOc1ccccc1NC(=O)c1ccc(C)c(S(=O)(=O)Nc2ccc(C)cc2)c1]

In some embodiments, the neuropilin antagonist is an inhibitor of neuropilin expression.

An “inhibitor of expression” refers to a natural or synthetic compound that has a biological effect to inhibit the expression of a gene. In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of NRP-1 mRNA by binding thereto and thus preventing protein translation or increasing mRNA degradation, thus decreasing the level of NRP-1, and thus activity, in a cell. For example, antisense oligonucleotides of at least about 15 bases and complementary to unique regions of the mRNA transcript sequence encoding NRP-1 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. NRP-1 gene expression can be reduced by contacting a patient or cell with a small double stranded RNA (dsRNA), or a vector or construct causing the production of a small double stranded RNA, such that NRP-1 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing NRP-1. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor of expression is an endonuclease. In a particular embodiment, the endonuclease is CRISPR-cas. In some embodiment, the endonuclease is CRISPR-cas9, which is from Streptococcus pyogenes. The CRISPR/Cas9 system has been described in U.S. Pat. No. 8,697,359 B1 and US 2014/0068797. In some embodiment, the endonuclease is CRISPR-Cpf1, which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. A therapeutically effective amount of a therapeutic compound may decrease tumour size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

The mode of administration selected will depend on the acuteness and severity of the condition being treated. Any mode of administration that produces desired therapeutic effect without unacceptable adverse effects is relevant in practicing the invention. Such modes of administration may include oral, rectal, topical, transdermal, sublingual, intramuscular, parenteral, intravenous, intracavity, vaginal, and adhesive matrix to be used during surgery. Vaginal administration of the neuropilin antagonist is also possible, for example by vaginal pessary or tablet, or by vaginal ring.

Typically, the drug of the present invention is administered to the subject in the form of a pharmaceutical composition, which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavouring or colouring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials.

In some embodiments, the neuropilin antagonist of the present invention is administered to the patient in combination with a progestogen.

As used herein, the term “progestogen” or “gestagen” has its general meaning in the art and covers synthetic hormone compounds which exert anti-estrogenic (counteracting the effects of estrogens in the body) and anti-gonadotropic (inhibiting the production of sex steroids and gonads) properties.

In some embodiments, the progestogen is selected from chlormadinone acetate, cyproterone acetate, desogestrel, dienogest, 5α-dihydroprogesterone, drospirenone (Yasmit®), ethanediol acetate, ethynodiol diacetate, etonouestrel (Nexplanon®), gestodene, 17-hydroxyprogesterone, levonorgestrel (Alesse®), medroxyprogesterone acetate (17α-hydroxy-6α-methylprogesterone acetate; Provera®), megestrol, megestrol acetate (17αacetoxy-6-dehydro-6-methylprogesterone), nestorone, nomegestrol acetate, norethindrone, norethindrone acetate (also known as norethindrone acetate), norethynodrel Enovid®), norgestimate, norgestrel, progesterone, tanaproget, trimegestone, pharmaceutically acceptable salts of any of the foregoing, and any combination thereof.

As used herein, the term “combination” is intended to refer to all forms of administration that provide a first drug together with a further (second, third . . . ) drug. The drugs may be administered simultaneous, separate or sequential and in any order. Drugs administered in combination have biological activity in the patient to which the drugs are delivered. Within the context of the invention, a combination thus comprises at least two different drugs, and wherein one drug is at least one neuropilin antagonist and wherein the other drug is at least one progestogen.

The invention will be further illustrated by the following figures and examples. However, these examples and figures should not be interpreted in any way as limiting the scope of the present invention.

### FIGURES

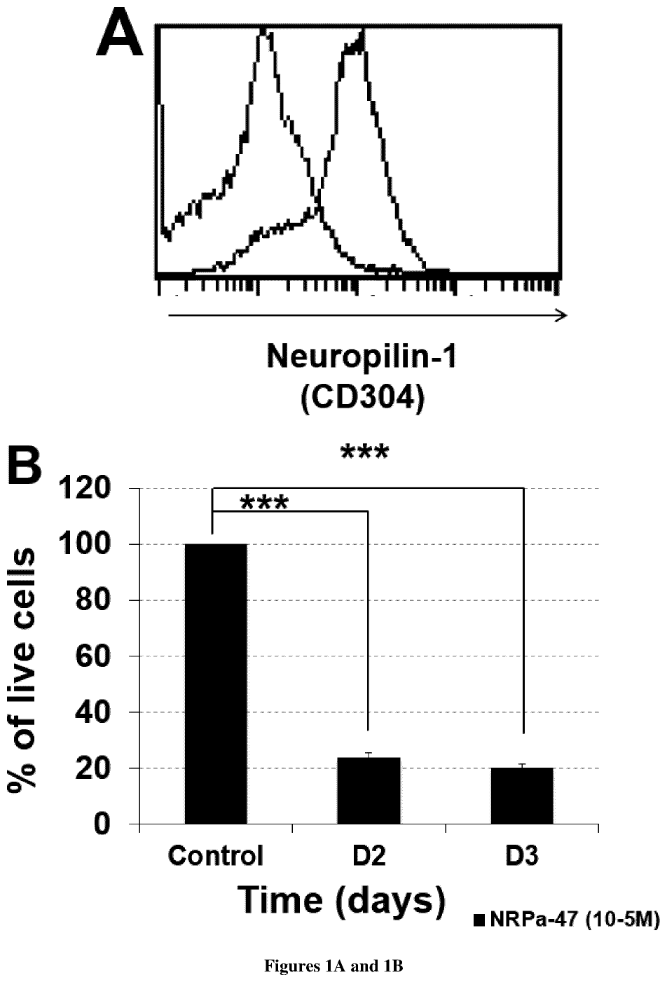

FIG. 1: Primary endometrial cell line (TH-EM1) express Neuropilin-1 and is sensitive to Neuropilin-1 inhibitor.
A—Histogram shows the expression and the high amount of Neuropilin-1 at the cell surface of the primary endometrial cell line.
B—Neuropilin antagonist (NRPa) such as NPRa-47 at 10⁻⁵M is significantly efficient to decrease 80% of cell survival in culture after 48 h of exposure (D2) and remains consistent at 72 h (D3). Data represent means±SD of 3 separate experiments, each. (***p<0.001).

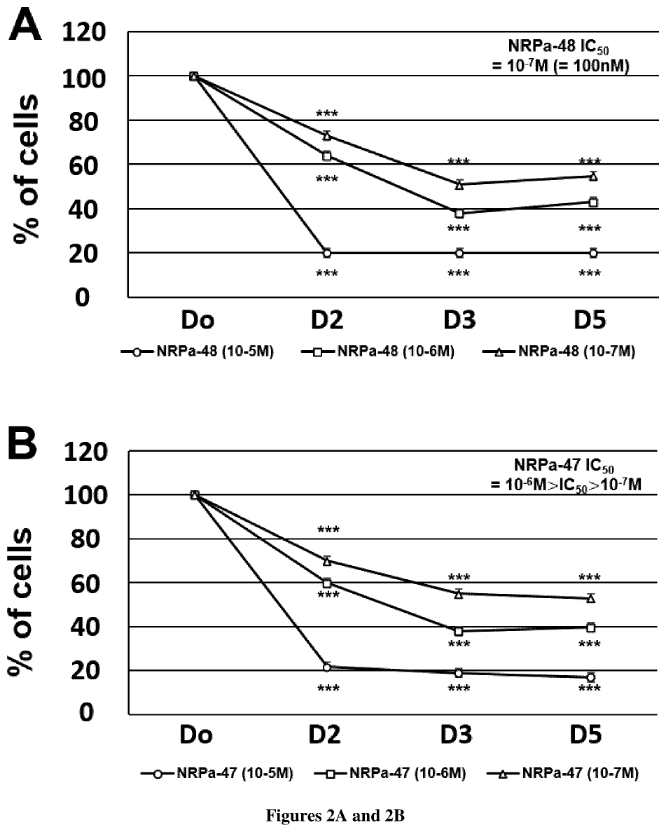

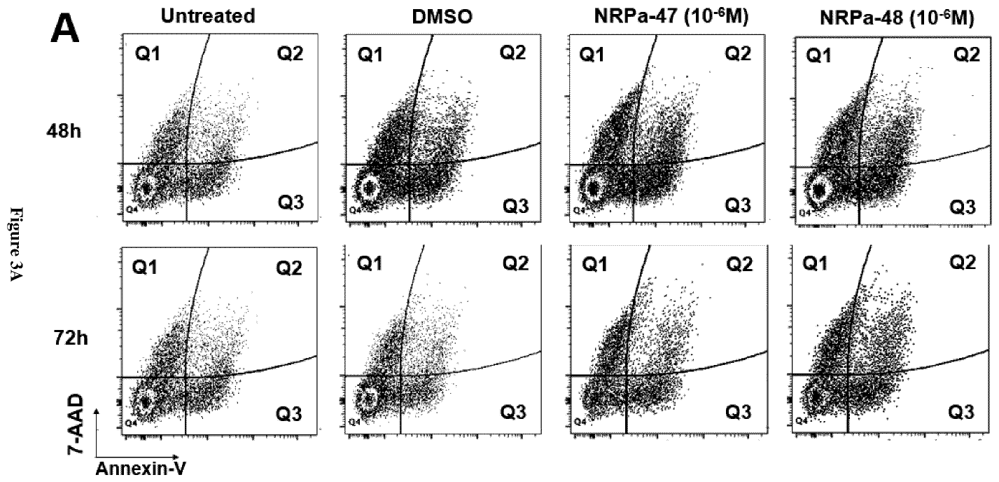

FIG. 2: Low concentration of NRPa is required to suppress primary endometrial cell proliferation.
A—A large concentration range of NRPa-48 is tested alone on primary endometrial cell proliferation. Calculated NRPa-48 IC₅₀ is closed to 10⁻⁷ M. Curves are representative of at least 3 separate experiments.
B—A large concentration range of NRPa-47 is tested alone on primary endometrial cell proliferation. Calculated NRPa-47 IC₅₀ is comprise between 10⁻⁶ to 10⁻⁷ M. Curves are representative of at least 3 separate experiments.
   Data represent means±SD. (***p<0.001).

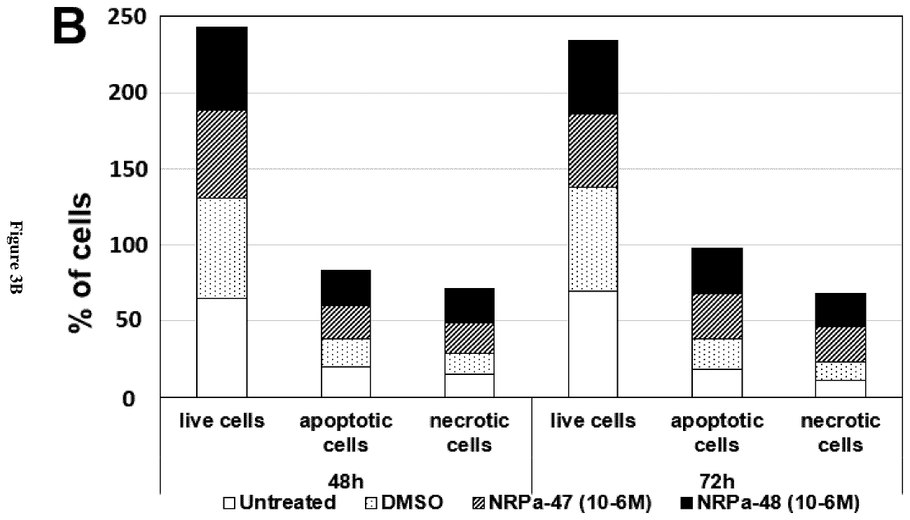

FIG. 3: NRPa induces apoptosis and necrosis of primary endometrial cell.
A—Dot plots show the induction of apoptosis (Annexin-V⁺/7-AAD⁺) and necrosis (Annexin-V⁻/7-AAD⁺) of endometrial cells induced by NRPa-47 and NRPa-48 at 10⁻⁶ M at 72 h compare to untreated and DMSO treated cells as control.
B—Histogram compared the ratio of live, apoptotic and necrotic endometrial cells under NRPa treatment.

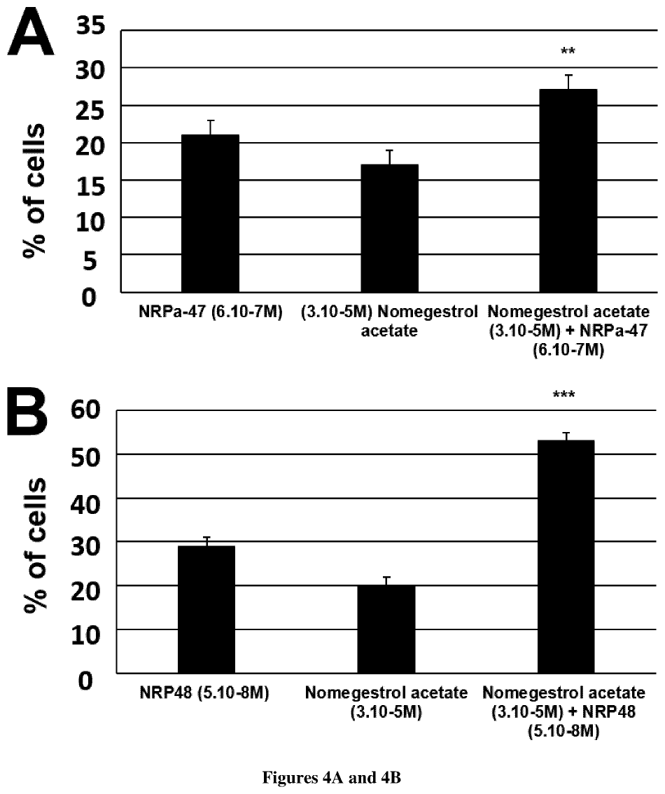

FIG. 4: NRPa and Progestatif drug association increases the inhibition of endometrial cell proliferation.
A—Histogram shows the percentage of growth inhibition of primary endometrial cells induced by Lutenyl® (Nomegestrol acetate) at 3·10⁻⁵ M, NRPa-47 (5·10⁻⁷M) alone or in association. Additif effect of the association is observed.
B—Histogram shows the percentage of growth inhibition of primary endometrial cells induced by Lutenyl® (Nomegestrol acetate) at 3·10^{-s} M, NRPa-48 (5·10⁻⁸M) alone or in association. Synergistic effect of the association is observed.
   Data represent means±SD. (**p<0.01, ***p<0.001).

### EXAMPLE

Methods

Cell Culture

The human primary unmodified endometrial cell line (TH-EM1) describes by Gogusev j et al. 2019 is cultured with Dulbecco modified Eagle medium supplemented with 10% of fetal calf serum (FCS), L-glutamine, and antibiotics (Invitrogen-Thermo Fisher, Cergy-Pontoise, France). Cells were plated in 96-well plates or in 12-well plates to study cell proliferation or apoptosis, respectively.

Flow Cytometry

Untreated proliferating TH-EM1 cells (2·10⁵ cells) were harvested and washed with PBS 1× with ca²⁺/mg²⁺ containing 2% FCS buffer. Then, cells were directly stained with phycoerythrin (PE)-Neuropilin-1 or irrelevant appropriate monoclonal antibodies as controlled using PBS 1×2% FCS (Miltenyi biotec, France). Then, stained cells were analyzed on a fluorescence activated cell sorter (FACS) Calibur flow cytometer (Becton Dickinson Co, Mountain View, Calif.) and data analysis was performed with Flowjo software.

Cell Proliferation Assay

Cells were plated in 200 μL/well in 96-well plates at 10⁴ cells/well and were treated or not with Neuropilin antagonists (NRPa) such as NRPa-47, NRPa-48 at several concentration (10⁻⁵ to 5·10⁻⁸M) alone or in association with different concentrations of progestatif drugs such as Lutenyl® (Nomegestrol acetate) at 3·10⁻⁵ M during 48 h, 72 h or 120 h. WST-1 (Roche®, France) was added for 2 h, then Optical Density was analyzed with a microplate reader (Microplate Manager 5.2, Bio-Rad) at 490 nm to determine the cell viability.

Apoptosis Assay

Cells were plated in 2000 μL/well in 12-well plates at 5·10⁵ cells/well and were treated or not with Neuropilin antagonists (NRPa) such as NRPa-47, NRPa-48 during 48 h to 72 h at 10⁻⁶M. Then, cells are harvested and stained using Annexin-V/7AAD kit protocol following manufacturer's recommendation. Then, stained cells were analyzed on a fluorescence activated cell sorter (FACS) Calibur flow cytometer (Becton Dickinson Co, Mountain View, Calif.) and data analysis was performed with Flowjo software.

Statistical Analysis:

Data are expressed as the arithmetic mean+/−SD of at least three different experiments. The statistical significance of results was evaluated by ANOVA, with probability values **p<0.01, ***p<0.001, being considered as significant.

Results

Neuropilin Expression and NRPa Sensitivity of Endometrial Cells

To test the efficiency of Neuropilin antagonist (NRPa) on endometriosis, we first examined its level expression on the human unmodified primary endometrial cell (Gogusev et al. 2019). As we show in FIG. 1A, Neuropilin (NRP-1) is significantly express at the cell surface of these cells. Even if NRP-1 is expressed, these might be or not sensitive to NRPa, thus we initially tested at high level concentration of NRPa (NRPa-47, 10⁻⁵M) if any effect is observed on cells. As expected, after 48 to 72 hours of exposure, quantity of endometrial cells is significantly decreased (80% of inhibition) (FIG. 1B).

Low Concentration of NRPa is Needed to Inhibit Endometrial Cell Growth.

To determine the sensitivity of endometrial cell to NRPa, several concentrations (10⁻⁵, 10⁻⁶, 10⁻⁷ M) were evaluated during a time course (including day-2 to day-5 examination). As expected, a rapid endometrial growth inhibition is obtained at high level concentration (10⁻⁵M) of NRPa (NRPa-47 and NRPa-48) since 2 days (D2) of exposure (FIGS. 2A and 2B). However, Low concentration of NRPa (NRPa-47 and NRPa-48) comprise between 10⁻⁶ and 10⁻⁷ M reached a maximum efficiency after 3 days (D3) of exposure since at D5 the effect stayed stable (FIGS. 2A and 2B). These results allowed us to determine the approximative IC₅₀ of NRPa-48 at 10⁻⁷ M (100 nM) and of NRPa-47 comprise between 10⁻⁶ to 10⁻⁷ M (FIGS. 2A and 2B).

Endometrial Cell Growth Inhibition is Mediated by the Apoptosis Induced by NRPa.

The endometrial cell apoptosis was followed under the NRPa exposure after 48 and 72 hours using Annexin-V/7-AAD staining. NRPa including NRPa-47 and NRPa-48, may induced endometrial cell death reaching a maximum at 72 hours of exposure (FIG. 3A). This induced cell death is divided in two steps, first the induction of apoptosis (Annexin-V⁻/7-AAD⁺ and Annexin-V⁻/7-AAD⁻ staining) and also the induction of necrosis (Annexin-V⁻/7-AAD⁺) (FIGS. 3A and 3B). Histogram clearly showed the percentage of each cell death phase induced by NRPa on endometrial cells at low concentration.

NRPa May Act Synergistically with Conventional Progestogen.

As patient suffering of endometriosis continuously received progestogen drugs, we investigated the effect of NRPa associated to it. Surprisingly, the association of low NRPa-47 concentration (6·10⁻⁷ M) with progestogen drug (Nomegestrol acetate) procured additive effect on cell growth inhibition (FIG. 4A). More importantly, using 2-fold less of NRPa-48 IC₅₀ concentration (5·10⁻⁸M) a synergistic effect is observed in presence of progestogen drug (FIG. 4B). These results reinforced the interest of the NRPa use in this pathology and strengthened the importance of its association of conventional progestogen drugs.

Discussion:

Taken together, this report highlighted the pivotal importance of the development of NRPa, which brought new tools for endometriosis treatment and for the knowledge of biological pathways involved in this disease. In this report, we observed that two structurally-related NRPa (NRPa-47 and NRPa-48) rapidly inhibit endometriosis cell line growth alone or in association with conventional hormonal treatment. In this context, NRPa may induce endometriosis cell line apoptosis. Future investigations are needed to identify the downstream pathway mediated by NRPa on kinase regulation as well as on the modulation of death receptors, pro-apoptotic and anti-apoptotic proteins.

In summary, NRPa might be used alone or in association with a conventional endometriosis hormonal treatment. This observation brought newest interest for the development of NRPa in this pathology.

### REFERENCES

Throughout this application, various references describe the state of the art to which this invention pertains. The disclosures of these references are hereby incorporated by reference into the present disclosure.
Gogusev J., Lepelletier Y., El Khattabi L., Grigoroiu M., and Validire P. Establishment and Characterization of a Stromal Cell Line Derived From a Patient With Thoracic Endometriosis. Reproductive Sciences, 2019.

1. A method of treating endometriosis in a patient in need thereof comprising administering to the patient a therapeutically effective amount of a neuropilin antagonist (NRPa).

2. The method of claim 1 wherein the patient suffers form peritoneal endometriosis, ovarian endometriosis, deep endometriosis or extrapelvic endometriosis.

3. The method of claim 1 wherein the subject is a human.

4. The method of claim 1 wherein the subject is a non-human mammal.

5. The method of claim 1 wherein the neuropilin antagonist is selected from the group consisting of antisense polynucleotides, interfering RNAs, catalytic RNAs, RNA-DNA chimeras, neuropilin-specific aptamers, anti-neuropilin antibodies, neuropilin-binding fragments of anti-neuropilin antibodies, neuropilin-binding small molecules, neuropilin-binding peptides, and other polypeptides that specifically bind neuropilin (including, but not limited to, neuropilin-binding fragments of one or more neuropilin ligands, optionally fused to one or more additional domains), such that the interaction between the neuropilin antagonist and neuropilin results in a reduction or cessation of neuropilin activity or expression.

6. The method of claim 1 wherein the neuropilin antagonist inhibits the interaction between a neuropilin protein (e.g. NRP-1) and its partners, in particular VEGF-A₁₆₅.

7. The method of claim 1 wherein the neuropilin antagonist is an antibody that specifically binds to a neuropilin (e.g. NRP-1 or NRP-2) and neutralizes its activity to activate neuropilin signalling pathway, and in particular inhibits the binding neuropilin and VEGF-A₁₆₅.

8. The method of claim 1 wherein the neuropilin antagonist is NRPa-47 or NRPa-48.

9. The method of claim 1 wherein the neuropilin antagonist is administered to the patient in combination with a progestogen.

10. The method of claim 1 wherein the progestogen is selected from chlormadinone acetate, cyproterone acetate, desogestrel, dienogest, 5α-dihydroprogesterone, drospirenone (Yasmit®), ethanediol acetate, ethynodiol diacetate, etonouestrel (Nexplanon®), gestodene, 17-hydroxyprogesterone, levonorgestrel (Alesse®), medroxyprogesterone acetate (17α-hydroxy-6α-methylprogesterone acetate; Provera®), megestrol, megestrol acetate (17αacetoxy-6-dehydro-6-methylprogesterone), nestorone, nomegestrol acetate, norethindrone, norethindrone acetate (also known as norethindrone acetate), norethynodrel Enovid®), norgestimate, norgestrel, progesterone, tanaproget, trimegestone, pharmaceutically acceptable salts of any of the foregoing, and any combination thereof.
